(19) United States
(12) Patent Application Publication
(10) Pub. No.: US 2024/0295542 A1
(43) Pub. Date: Sep. 5, 2024

(54) In-Situ Mechanical Characterization Of Cement Sheath Exposed To Chemical Species

(71) Applicant: Halliburton Energy Services, Inc., Houston, TX (US)

(72) Inventors: Gunnar Lende, Stavanger (NO); Siva Rama Krishna Jandhyala, Katy, TX (US); Giorgio DeVera, Houston, TX (US)

(73) Assignee: Halliburton Energy Services, Inc., Houston, TX (US)

(21) Appl. No.: 18/116,224

(22) Filed: Mar. 1, 2023

(51) Int. Cl. G01N 33/38; C04B 28/02; C04B 40/02; C09K 8/46; E21B 47/005; G01V 1/50

(52) U.S. Cl. CPC G01N 33/383; C04B 28/02; C04B 40/0231; C09K 8/46; E21B 47/005; G01V 1/50; C04B 2111/00706; E21B 33/14

(57) ABSTRACT

A method may include: preparing a plurality of cement slurries, wherein the plurality of cement slurries each comprise a cement and volume fraction of water; curing the plurality of cement slurries to form a plurality of set cement samples; exposing the plurality of set cement samples to a chemical species; allowing the chemical species to at least partially modify the plurality of set cement samples to form a plurality of composite cement samples; measuring a dynamic physical property of each of the plurality of composite cement samples to generate a dynamic physical property dataset; measuring a static physical property of each of the plurality of composite cement samples to generate a static physical property dataset; and correlating the static physical property dataset as a function of the dynamic physical property dataset and volume fraction of water to generate a composite cement property model.

### BACKGROUND

In well cementing, such as well construction and remedial cementing, cement compositions are commonly utilized. Cement slurries may be used in a variety of subterranean applications. For example, in subterranean well construction, a pipe string (e.g., casing, liners, expandable tubulars, etc.) may be run into a well bore and cemented in place. The process of cementing the pipe string in place is commonly referred to as “primary cementing.” In a typical primary cementing method, a cement slurry or a resin-based system may be pumped into an annulus between the walls of the well bore and the exterior surface of the pipe string disposed therein. The cement slurry or resin system may set in the annular space, thereby forming an annular sheath of hardened, substantially impermeable cement or resin (i.e., a cement sheath or annular sealant) that may support and position the pipe string in the well bore and may bond the exterior surface of the pipe string to the subterranean formation. Among other things, the cement sheath surrounding the pipe string functions to prevent the migration of fluids in the annulus, as well as protecting the pipe string from corrosion. Cement slurries also may be used in remedial cementing methods, for example, to seal cracks or holes in pipe strings or cement sheaths, to seal highly permeable formation zones or fractures, to place a cement plug, and the like.

In wellbores where carbon dioxide (CO₂) is present, such as in carbon capture underground storage (CCUS) wells, or any other corrosive fluids are present, there may be additional considerations for the long-term integrity of the cement sheath. For instance, a cement sheath which is exposed to CO₂ may begin to uptake the CO₂ into the matrix of the cement which may cause chemical and physical changes within the cement. Cement exposed to CO₂ behaves like a composite material with spatially varying mechanical properties such as Young's modulus (YM), Poisson's ratio (PR), and unconfined compressive strength (UCS). The carbonated portion of the cement may have different mechanical properties than the uncarbonated portion which may affect the capability of the cement to sustain zonal isolation in the wellbore. As the extent of carbonation evolves over time, the ability of the cement sheath to sustain zonal isolation may also be time dependent. The same is true for any corrosive fluids, other than CO₂, that tend to attack hardened cement and cause changes to its properties. The following paragraphs use CO₂ as an example to describe the work method. However, the method is the same in case of any other corrosive gases.

One challenge in assessing in-situ zonal isolation of a cement sheath within a wellbore, or in a lab, is the lack of measurement techniques for continuous measurement of spatially varying mechanical properties at different CO₂ exposure conditions. A common technique for assessing zonal isolation is to deploy a coring tool into the wellbore to take a core sample of a cement sheath which is then analyzed in a laboratory. The coring technique is slow and leaves a defect in the cement sheath which must be repaired through remedial cementing techniques.

Another challenge is evaluating cement designs for use in wellbores with CO₂ exposure. There exist numerical simulators for life of the well modeling of cements which can predict the stresses a cement sheath may experience over a period of time to evaluate if the cement sheath is likely to fail to maintain zonal isolation. However, in carbon dioxide containing wells, the time-evolved mechanical properties of carbonated cement add additional degrees of freedom which may not be adequately captured by numerical simulators.

### BRIEF DESCRIPTION OF THE DRAWINGS

These drawings illustrate certain aspects of some of the embodiments of the present disclosure and should not be used to limit or define the disclosure.

FIG. 1 is a cross sectional view of a partially carbonated cement plug sample.

FIG. 2 is a block flow diagram which illustrates a process to build a composite cement property model in accordance with some embodiments of the present disclosure.

FIG. 3 is a block flow diagram which illustrates a method of using a composite cement property model in accordance with some embodiments of the present disclosure.

FIG. 4 is a schematic illustration of introduction of a cement slurry into a wellbore, in accordance with some embodiments of the present disclosure.

FIG. 5 is a plot of the static Young's modulus to dynamic Young's modulus for a cement in accordance with some embodiments of the present disclosure.

FIG. 6 is a plot of the static Poisson's ratio to the dynamic Poisson's ratio for a cement in accordance with some embodiments of the present disclosure.

FIG. 7 is a parity plot of a composite carbonated cement Young's modulus property model in accordance with some embodiments of the present disclosure.

FIG. 8 is a parity plot of the composite carbonated cement Poisson's ratio property model in accordance with some embodiments of the present disclosure.

### DETAILED DESCRIPTION

The present disclosure may generally relate to in-situ evaluation of composite cement sheaths where the composite cement sheath comprises an unmodified portion and a chemically modified portion. More particularly, embodiments may be directed to methods using a composite cement property model to correlate a dynamic measurement of a cement physical property to an equivalent static property. Cement physical properties such as Young's modulus are parameters utilized when evaluating stress and strain in a cement. There are two main measurement methods for determining cement physical properties including static methods which are based on a physical deformation of a cement sample with a known force, and dynamic methods typically based on ultrasonic wave velocities. While the physical properties measured by dynamic and static methods should coincide, oftentimes the measured values are different due to inelastic effects within a cement sample, among other factors. In embodiments with a composite cement sheath which has been modified by chemical species, the physical properties measured by dynamic and static methods may further diverge than in non-composite cements.

The composite cement property model disclosed herein more accurately predicts the static physical properties of a composite cement from the equivalent dynamic physical properties. The composite cement property model also improves the function of numerical wellbore simulators by more accurately predicting the time-evolved mechanical properties of composite cement. By combining a composite cement property model with a numerical simulator, a more accurate assessment of a cement slurry may be provided by the numerical simulator. For example, the numerical simulator may more accurately predict if the well integrity is maintained throughout the desired life of the well.

As discussed above, cement exposed to carbon dioxide may become carbonated to form a chemically modified portion of cement. The physical and chemical properties of the chemically modified carbonated portion of the cement may be disparate from an unmodified portion of the cement. While the methods disclosed herein may be directed to chemical modification by carbon dioxide, the methods are applicable to any other chemical species which modifies the chemical properties of cement including, but not limited to, carbon dioxide, ammonia, hydrogen sulfide (H₂S), and acids, for example. Any of the foregoing species may chemically alter a cement thereby forming a composite cement.

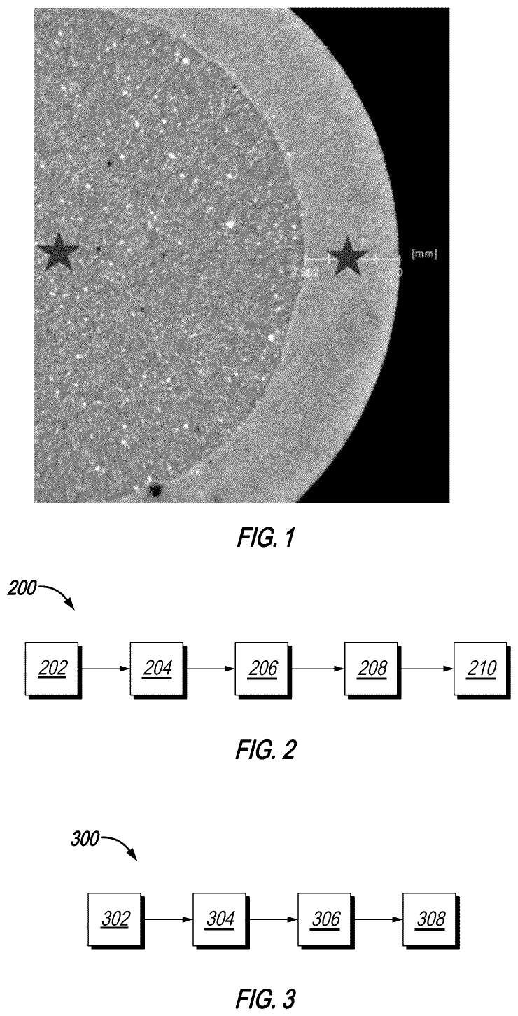

FIG. 1 is a cross sectional view of a partially carbonated cement sample. In FIG. 1, the darker inner core is uncarbonated cement and the outer lighter shell is carbonated cement. One method of determining the dynamic physical properties of the partially carbonated cement sample is to place ultrasonic transducers such as a transmitter and receiver, indicated as stars in FIG. 1, on a flat surface of the partially carbonated cement and measuring the travel time of an ultrasonic pulse between the transmitter and receiver. In embodiments, the transmitter and receiver may be placed on the partially carbonated cement to operate in a direct transmission mode, in a semi-direct transmission mode where the transmitter and receiver are placed orthogonally relative to each other, or in an indirect or surface transmission mode. Alternatively, a single transceiver transducer and a reflector may be used to transmit and capture the ultrasonic pulse. The results of the ultrasonic testing may be used as input to algorithms, methods, and models well known in the art to determine a dynamic cement physical property. The American Petroleum Institute (API) publishes API RP 10B-2 (Recommended Practice for Testing Well Cements), with the latest version being dated Apr. 1, 2013, which outlines the standard methods testing of cement slurries and set cements to determining cement physical properties of each.

The composite cement property model predicts static cement physical properties of a composite cement as a function of measured dynamic physical properties and composition of cement, for example the volume fraction of water used to prepare the cement. Static cement physical properties are properties which are measured by destructive testing of a cement. Some examples of destructive testing may include, without limitation, crushing tests (in accordance with ASTM D7012-10) in the presence or absence of confined pressure, pull out tests (in accordance with ASTM C900-19), hardness tests (in accordance with ASTM D785 and/or ASTM E10), and scratch resistance tests in accordance with (ISO 4586). Dynamic cement physical properties are properties which are measured by non-destructive methods, typically using sonic or ultrasonic waves in compression and or shear mode. Dynamic physical properties of a set cement may be measured by any suitable method including ultrasonic pulse velocity methods, acoustic methods, and flat-jack test methods, for example. The destructive testing and/or non-destructive testing can be carried out according to any applicable ASTM and/or API methods. Static and dynamic properties may include any property of a set cement, including, but not limited to, Young's modulus, Poisson's ratio, unconfined compressive strength, tensile strength, flexural strength, bulk modulus, shear modulus and shear strength, for example. Equation 1 is a generalized composite cement property model which shows the relationship between the static property as a function of the several parameters including measured dynamic property and volume fraction of water, cement composition, pressure, and temperature.

static physical property=f(dynamic physical property,volume fraction of water, cement composition,Pressure,Temperature) Equation 1

A composite cement property model may be built for each type of dynamic physical property and corresponding static physical property, where the composite cement property model is specific to each chemical species or species which has chemically modified the composite cement. In embodiments, a composite cement property model may include a composite cement Young's modulus property model corresponding to a carbonated cement. In further embodiments, a composite cement property model may include a composite cement Poisson's ratio property model corresponding to an H₂S modified cement. In further embodiments, the composite cement property model may model a composite cement property selected from Young's modulus, Poisson's ratio, unconfined compressive strength, tensile strength, flexural strength, bulk modulus, shear modulus and shear strength, and a chemical species selected from carbon dioxide, ammonia, hydrogen sulfide (H₂S), and acid.

FIG. 2 is a block flow diagram which illustrates a process 200 to build a composite cement property model. Process 200 begins at block 202. In block 202, a plurality of cement slurries are prepared where the plurality of cement slurries comprise cement and varying composition, and volume fractions of water. After preparation, the cement slurries are allowed to cure to form a plurality of set cement samples. The curing step can be performed at atmospheric pressure and room temperature or may be performed at elevated pressure and temperatures. For example, the curing can be performed at a temperature in a range of from about 20° C. to about 200° C. Alternatively, at a temperature in a range of about 20° C. to about 50° C., about 50° C. to about 100° C., about 100° C. to about 150° C., about 150° C. to about 200° C., or any ranges therebetween. The curing can be performed at a pressure in a range of about 1 bar to about 1500 bar. Alternatively, at a pressure in a range of about 1 bar to about 10 bar, about 10 bar to about 100 bar, about 100 bar to about 500 bar, about 500 bar to about 1000 bar, about 1000 bar to about 1500 bar, or any ranges therebetween.

From block 202, process 200 proceeds to block 204 where the plurality of set cement samples from block 202 are subjected to chemical modification by a chemical species, such as those chemical species previously discussed to from a plurality of composite cement samples. The plurality of set cement samples may be subjected to chemical modification by any suitable means, including, but not limited to submersion in a solution containing a chemical specie(s) and/or exposure to a gas containing the chemical specie(s), for example. The plurality of set cement samples may be subjected to the chemical modification for any suitable period of time, such as in a range of about 1 day to about 1 year or greater. Alternatively, from about 1 day to about 1 week, from about 1 week to about 1 month, about 1 month to about 6 months, about 6 months to about 1 year, about 1 year to about 10 years, or any ranges of time therebetween. To capture the effects of different extent of chemical modification, at least some of the set cement samples may be subjected to chemical modification for differing amounts of time.

From block 204, process 200 proceeds to block 206 where the plurality of composite cement samples from block 204 are subjected to non-destructive testing to determine at least one dynamic physical property such as dynamic Young's modulus, dynamic Poisson's ratio, dynamic unconfined compressive strength, dynamic tensile strength, dynamic flexural strength, dynamic modulus of elasticity, and dynamic shear strength, for example. Non-destructive testing may include any of the previously disclosed methods such as ultrasonic pulse velocity methods, acoustic methods and flat-jack test methods.

From block 206, process 200 proceeds to block 208 where the plurality of composite cement samples from block 204 are subjected to destructive testing to determine at least one static physical property such as static Young's modulus, static Poisson's ratio, static unconfined compressive strength, static tensile strength, static flexural strength, static modulus of elasticity, and static shear strength, for example, where the at least one static physical property corresponds to the dynamic physical property from block 206. Destructive testing may include crushing methods, pull out testing, hardness tests, scratch resistance tests.

From block 208, process 200 proceeds to block 210 where the static physical property data from block 206 is as a function of the dynamic physical property data from block 204 and the volume fractions of water, and optionally composition, from block 202 to form the composite cement property model. The composite cement property model may be specific to the type cement selected in block 202 as well as the chemical modification in block 204. The data from block 202, 206, and 208 may be fit to a model of any suitable form, including, but not limited to, linear, multilinear, parabolic, exponential, derivative, integral, hyperbolic, trigonometric, and combinations thereof. The model may be fit using any suitable technique such as regression, for example. Alternately, the model may be a black box model, including, but not limited to, artificial neural network, convolutional neural network, recurrent neural network, decision tree, random forest, machine learning boosting, extreme gradient boosting, Gaussian process regression, spline regression, and multi-variate adaptive regression spline, for example. The fit model from block 210 is the composite cement property model.

Equation 2 is a generalized multilinear form of a composite cement property model where β is the slope coefficient for each factor from dataset from data from block 202, 206, and 208 included in the model fit analysis in block 210.

$$static physical property = f ( {β}_{0} + {β}_{1} {Factor}_{1} + {β}_{2} {Factor}_{2} + … + {β}_{n} {Factor}_{n} ) \qquad Equation 2$$

FIG. 3 illustrates a process 300 of using a composite cement property model. Process 300 begins with block 302 where cement plan data is defined. The cement plan data may include, without limitation, geographic region, wellbore job type, planned pumping rates, planned cement composition, planned spacer composition, planned fluid volumes, planned fluid densities, specifics of the construction of the well such as presence of bottom plugs, well true vertical depth (TVD), inclination, and standoff value, specifics about the subterranean formation including, but not limited to, pore pressure, fracture gradient, bottomhole static temperature, wellbore temperature profile, flow potential factor, and chemical species in the wellbore which may modify the chemical properties of cement, and cement composition. From block 302, process 300 proceeds to block 304 where the cement composition from block 302 is prepared and dynamic mechanical properties of the cement composition are measured as a function of time. From block 304, process 300 proceeds to block 306 where static mechanical properties of the cement composition are predicted as a function of time using the measured dynamic mechanical properties from block 304 and the composition of cement and volume fraction of water selected in block 302. From block 306, process 300 proceeds to block 308 where the predicted static mechanical properties from block 306 and well information from block 302 are used in numerical simulators to predict whether the cement sheath will withstand the mechanical loads exerted on it. If the cement sheath is calculated as surviving, the composition identified in block 302 is selected and pumped into a subterranean formation as part of a wellbore cementing operation. If not, the composition is modified and the process 300 is repeated until the cement composition selected in block 302 is determined to remain mechanically intact for the loads exerted on the composite cement sheath.

Cement compositions described herein may generally include a hydraulic cement and water. A variety of hydraulic cements may be utilized in accordance with the present disclosure, including, but not limited to, those comprising calcium, aluminum, silicon, oxygen, iron, and/or sulfur, which set and harden by reaction with water. Suitable hydraulic cements may include, but are not limited to, Portland cements, pozzolana cements, gypsum cements, high alumina content cements, silica cements, and any combination thereof. In certain examples, the hydraulic cement may include a Portland cement. In some examples, the Portland cements may include Portland cements that are classified as Classes A, C, H, and G cements according to American Petroleum Institute, API Specification for Materials and Testing for Well Cements, API Specification 10, Fifth Ed., Jul. 1, 1990. In addition, hydraulic cements may include cements classified by American Society for Testing and Materials (ASTM) in C150 (Standard Specification for Portland Cement), C595 (Standard Specification for Blended Hydraulic Cement) or C1157 (Performance Specification for Hydraulic Cements) such as those cements classified as ASTM Type I, II, or III. The hydraulic cement may be included in the cement composition in any amount suitable for a particular composition. Without limitation, the hydraulic cement may be included in the cement compositions in an amount in the range of from about 10% to about 80% by weight of dry blend in the cement composition. For example, the hydraulic cement may be present in an amount ranging between any of and/or including any of about 10%, about 15%, about 20%, about 25%, about 30%, about 35%, about 40%, about 45%, about 50%, about 55%, about 60%, about 65%, about 70%, about 75%, or about 80% by weight of the cement compositions.

The water may be from any source provided that it does not contain an excess of compounds that may undesirably affect other components in the cement compositions. For example, a cement composition may include fresh water or saltwater. Saltwater generally may include one or more dissolved salts therein and may be saturated or unsaturated as desired for a particular application. Seawater or brines may be suitable for use in some examples. Further, the water may be present in an amount sufficient to form a pumpable slurry. In certain examples, the water may be present in the cement composition in an amount in the range of from about 33% to about 200% by weight of the cementitious materials. For example, the water cement may be present in an amount ranging between any of and/or including any of about 33%, about 50%, about 75%, about 100%, about 125%, about 150%, about 175%, or about 200% by weight of the cementitious materials. The cementitious materials referenced may include all components which contribute to the compressive strength of the cement composition such as the hydraulic cement and supplementary cementitious materials, for example.

As mentioned above, the cement composition may include supplementary cementitious materials. The supplementary cementitious material may be any material that contributes to the desired properties of the cement composition. Some supplementary cementitious materials may include, without limitation, fly ash, blast furnace slag, silica fume, pozzolans, kiln dust, and clays, for example.

The cement composition may include kiln dust as a supplementary cementitious material. “Kiln dust,” as that term is used herein, refers to a solid material generated as a by-product of the heating of certain materials in kilns. The term “kiln dust” as used herein is intended to include kiln dust made as described herein and equivalent forms of kiln dust. Depending on its source, kiln dust may exhibit cementitious properties in that it can set and harden in the presence of water. Examples of suitable kiln dusts include cement kiln dust, lime kiln dust, and combinations thereof. Cement kiln dust may be generated as a by-product of cement production that is removed from the gas stream and collected, for example, in a dust collector. Usually, large quantities of cement kiln dust are collected in the production of cement that are commonly disposed of as waste. The chemical analysis of the cement kiln dust from various cement manufactures varies depending on a number of factors, including the particular kiln feed, the efficiencies of the cement production operation, and the associated dust collection systems. Cement kiln dust generally may include a variety of oxides, such as SiO₂, Al₂O₃, Fe₂O₃, CaO, MgO, SO₃, Na₂O, and K₂O. The chemical analysis of lime kiln dust from various lime manufacturers varies depending on several factors, including the particular limestone or dolomitic limestone feed, the type of kiln, the mode of operation of the kiln, the efficiencies of the lime production operation, and the associated dust collection systems. Lime kiln dust generally may include varying amounts of free lime and free magnesium, lime stone, and/or dolomitic limestone and a variety of oxides, such as SiO₂, Al₂O₃, Fe₂O₃, CaO, MgO, SO₃, Na₂O, and K₂O, and other components, such as chlorides. A cement kiln dust may be added to the cement composition prior to, concurrently with, or after activation. Cement kiln dust may include a partially calcined kiln feed which is removed from the gas stream and collected in a dust collector during the manufacture of cement. The chemical analysis of CKD from various cement manufactures varies depending on a number of factors, including the particular kiln feed, the efficiencies of the cement production operation, and the associated dust collection systems. CKD generally may comprise a variety of oxides, such as SiO₂, Al₂O₃, Fe₂O₃, CaO, MgO, SO₃, Na₂O, and K₂O. The CKD and/or lime kiln dust may be included in examples of the cement composition in an amount suitable for a particular application.

In some examples, the cement composition may further include one or more of slag, natural glass, shale, amorphous silica, or metakaolin as a supplementary cementitious material. Slag is generally a granulated, blast furnace by-product from the production of cast iron including the oxidized impurities found in iron ore. The cement may further include shale. A variety of shales may be suitable, including those including silicon, aluminum, calcium, and/or magnesium. Examples of suitable shales include vitrified shale and/or calcined shale. In some examples, the cement composition may further include amorphous silica as a supplementary cementitious material. Amorphous silica is a powder that may be included in embodiments to increase cement compressive strength.

In some examples, the cement composition may further include a variety of fly ashes as a supplementary cementitious material which may include fly ash classified as Class C, Class F, or Class N fly ash according to American Petroleum Institute, API Specification for Materials and Testing for Well Cements, API Specification 10, Fifth Ed., Jul. 1, 1990. In some examples, the cement composition may further include zeolites as supplementary cementitious materials. Zeolites are generally porous alumino-silicate minerals that may be either natural or synthetic. Synthetic zeolites are based on the same type of structural cell as natural zeolites and may comprise aluminosilicate hydrates. As used herein, the term “zeolite” refers to all natural and synthetic forms of zeolite.

Where used, one or more of the aforementioned supplementary cementitious materials may be present in the cement composition. For example, without limitation, one or more supplementary cementitious materials may be present in an amount of about 0.1% to about 80% by weight of the cement composition. For example, the supplementary cementitious materials may be present in an amount ranging between any of and/or including any of about 0.1%, about 10%, about 20%, about 30%, about 40%, about 50%, about 60%, about 70%, or about 80% by weight of the cement.

In some examples, the cement composition may further include hydrated lime. As used herein, the term “hydrated lime” will be understood to mean calcium hydroxide. In some embodiments, the hydrated lime may be provided as quicklime (calcium oxide) which hydrates when mixed with water to form the hydrated lime. The hydrated lime may be included in examples of the cement composition, for example, to form a hydraulic composition with the supplementary cementitious components. For example, the hydrated lime may be included in a supplementary cementitious material-to-hydrated-lime weight ratio of about 10:1 to about 1:1 or 3:1 to about 5:1. Where present, the hydrated lime may be included in the set cement composition in an amount in the range of from about 10% to about 100% by weight of the cement composition, for example. In some examples, the hydrated lime may be present in an amount ranging between any of and/or including any of about 10%, about 20%, about 40%, about 60%, about 80%, or about 100% by weight of the cement composition. In some examples, the cementitious components present in the cement composition may consist essentially of one or more supplementary cementitious materials and the hydrated lime. For example, the cementitious components may primarily comprise the supplementary cementitious materials and the hydrated lime without any additional components (e.g., Portland cement, fly ash, slag cement) that hydraulically set in the presence of water.

Lime may be present in the cement composition in several; forms, including as calcium oxide and or calcium hydroxide or as a reaction product such as when Portland cement reacts with water. Alternatively, lime may be included in the cement composition by amount of silica in the cement composition. A cement composition may be designed to have a target lime to silica weight ratio. The target lime to silica ratio may be a molar ratio, molal ratio, or any other equivalent way of expressing a relative amount of silica to lime. Any suitable target time to silica weight ratio may be selected including from about 10/90 lime to silica by weight to about 40/60 lime to silica by weight. Alternatively, about 10/90 lime to silica by weight to about 20/80 lime to silica by weight, about 20/80 lime to silica by weight to about 30/70 lime to silica by weight, or about 30/70 lime to silica by weight to about 40/63 lime to silica by weight.

Other additives suitable for use in subterranean cementing operations also may be included in embodiments of the cement composition. Examples of such additives include, but are not limited to: weighting agents, lightweight additives, gas-generating additives, mechanical-property-enhancing additives, lost-circulation materials, filtration-control additives, fluid-loss-control additives, defoaming agents, foaming agents, thixotropic additives, and combinations thereof. In embodiments, one or more of these additives may be added to the cement composition after storing but prior to the placement of a cement composition into a subterranean formation. In some examples, the cement composition may further include a dispersant. Examples of suitable dispersants include, without limitation, sulfonated-formaldehyde-based dispersants (e.g., sulfonated acetone formaldehyde condensate) or polycarboxylated ether dispersants. In some examples, the dispersant may be included in the cement composition in an amount in the range of from about 0.01% to about 5% by weight of the cementitious materials. In specific examples, the dispersant may be present in an amount ranging between any of and/or including any of about 0.01%, about 0.1%, about 0.5%, about 1%, about 2%, about 3%, about 4%, or about 5% by weight of the cementitious materials.

In some examples, the cement composition may further include a set retarder. A broad variety of set retarders may be suitable for use in the cement compositions. For example, the set retarder may comprise phosphonic acids, such as ethylenediamine tetra(methylene phosphonic acid), diethylenetriamine penta(methylene phosphonic acid), etc.; lignosulfonates, such as sodium lignosulfonate, calcium lignosulfonate, etc.; salts such as stannous sulfate, lead acetate, monobasic calcium phosphate, organic acids, such as citric acid, tartaric acid, etc.; cellulose derivatives such as hydroxyl ethyl cellulose (HEC) and carboxymethyl hydroxyethyl cellulose (CMHEC); synthetic co- or ter-polymers comprising sulfonate and carboxylic acid groups such as sulfonate-functionalized acrylamide-acrylic acid co-polymers; borate compounds such as alkali borates, sodium metaborate, sodium tetraborate, potassium pentaborate; derivatives thereof, or mixtures thereof. Examples of suitable set retarders include, among others, phosphonic acid derivatives. Generally, the set retarder may be present in the cement composition in an amount sufficient to delay the setting for a desired time. In some examples, the set retarder may be present in the cement composition in an amount in the range of from about 0.01% to about 10% by weight of the cementitious materials. In specific examples, the set retarder may be present in an amount ranging between any of and/or including any of about 0.01%, about 0.1%, about 1%, about 2%, about 4%, about 6%, about 8%, or about 10% by weight of the cementitious materials.

In some examples, the cement composition may further include an accelerator. A broad variety of accelerators may be suitable for use in the cement compositions. For example, the accelerator may include, but are not limited to, aluminum sulfate, alums, calcium chloride, calcium nitrate, calcium nitrite, calcium formate, calcium sulphoaluminate, calcium sulfate, gypsum-hemihydrate, sodium aluminate, sodium carbonate, sodium chloride, sodium silicate, sodium sulfate, ferric chloride, or a combination thereof. In some examples, the accelerators may be present in the cement composition in an amount in the range of from about 0.01% to about 10% by weight of the cementitious materials. In specific examples, the accelerators may be present in an amount ranging between any of and/or including any of about 0.01%, about 0.1%, about 1%, about 2%, about 4%, about 6%, about 8%, or about 10% by weight of the cementitious materials.

Cement compositions generally should have a density suitable for a particular application. By way of example, the cement composition may have a density in the range of from about 8 pounds per gallon (“ppg”) (959 kg/m³) to about 20 ppg (2397 kg/m³), or about 8 ppg to about 12 ppg (1437. kg/m³), or about 12 ppg to about 16 ppg (1917.22 kg/m³), or about 16 ppg to about 20 ppg, or any ranges therebetween. Examples of the cement compositions may be foamed or unfoamed or may comprise other means to reduce their densities, such as hollow microspheres, low-density elastic beads, or other density-reducing additives known in the art.

The cement slurries disclosed herein may be used in a variety of subterranean applications, including primary and remedial cementing. The cement slurries may be introduced into a subterranean formation and allowed to set. In primary cementing applications, for example, the cement slurries may be introduced into the annular space between a conduit located in a wellbore and the walls of the wellbore (and/or a larger conduit in the wellbore), wherein the wellbore penetrates the subterranean formation. The cement slurry may be allowed to set in the annular space to form an annular sheath of hardened cement. The cement slurry may form a barrier that prevents the migration of fluids in the wellbore. The cement composition may also, for example, support the conduit in the wellbore. In remedial cementing applications, the cement compositions may be used, for example, in squeeze cementing operations or in the placement of cement plugs. By way of example, the cement compositions may be placed in a wellbore to plug an opening (e.g., a void or crack) in the formation, in a gravel pack, in the conduit, in the cement sheath, and/or between the cement sheath and the conduit (e.g., a micro annulus).

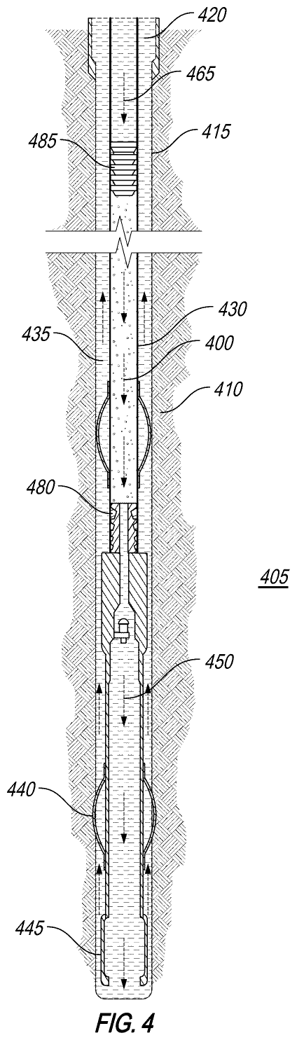

Reference is now made to FIG. 4, illustrating use of a cement slurry 400. Cement slurry 400 may comprise any of the components described herein and may be designed using a composite cement property model as described above. Cement slurry 400 may be placed into a subterranean formation 405 in accordance with example systems, methods and cement slurries. The subterranean formation 405 may a reservoir for oil, gas, and water or may be a depleted formation. In embodiments the subterranean formation 405 is used for carbon capture underground storage. As illustrated, a wellbore 410 may be drilled into the subterranean formation 405. While wellbore 410 is shown extending generally vertically into the subterranean formation 405, the principles described herein are also applicable to wellbores that extend at an angle through the subterranean formation 405, such as horizontal and slanted wellbores. As illustrated, the wellbore 410 comprises walls 415. In the illustration, casing 430 may be cemented to the walls 415 of the wellbore 410 by cement sheath 420. In the illustration, one or more additional conduits (e.g., intermediate casing, production casing, liners, etc.), shown here as casing 430 may also be disposed in the wellbore 410. As illustrated, there is a wellbore annulus 435 formed between the casing 430 and the walls 415 of the wellbore 410. One or more centralizers 440 may be attached to the casing 430, for example, to centralize the casing 430 in the wellbore 410 prior to and during the cementing operation. The cement slurry 400 may be pumped down the interior of the casing 430. The cement slurry 400 may be allowed to flow down the interior of the casing 430 through the casing shoe 445 at the bottom of the casing 430 and up around the casing 430 into the wellbore annulus 435. The cement slurry 400 may be allowed to set in the wellbore annulus 435, for example, to form a cement sheath that supports and positions the casing 430 in the wellbore 410. While not illustrated, other techniques may also be utilized for introduction of the cement slurry 400. By way of example, reverse circulation techniques may be used that include introducing the cement slurry 400 into the subterranean formation 405 by way of the wellbore annulus 435 instead of through the casing 430. As it is introduced, the cement slurry 400 may displace other fluids 450, such as drilling fluids and/or spacer fluids that may be present in the interior of the casing 430 and/or the wellbore annulus 435. While not illustrated, at least a portion of the displaced fluids 450 may exit the wellbore annulus 435 via a flow line and be deposited, for example, in one or more retention pits. A bottom plug 455 may be introduced into the wellbore 410 ahead of the cement slurry 400, for example, to separate the cement slurry 400 from the fluids 450 that may be inside the casing 430 prior to cementing. After the bottom plug 455 reaches the landing collar 480, a diaphragm or other suitable device should rupture to allow the cement slurry 400 through the bottom plug 455. The bottom plug 455 is shown on the landing collar 480. In the illustration, a top plug 485 may be introduced into the wellbore 410 behind the cement slurry 400. The top plug 460 may separate the cement slurry 400 from a displacement fluid 465 and also push the cement slurry 400 through the bottom plug 455.

The following statements may describe certain embodiments of the disclosure but should be read to be limiting to any particular embodiment.

Statement 1. A method comprising: preparing a plurality of cement slurries, wherein the plurality of cement slurries each comprise a cement and volume fraction of water; curing the plurality of cement slurries to form a plurality of set cement samples; exposing the plurality of set cement samples to a chemical species; allowing the chemical species to at least partially modify the plurality of set cement samples to form a plurality of composite cement samples; measuring a dynamic physical property of each of the plurality of composite cement samples to generate a dynamic physical property dataset; measuring a static physical property of each of the plurality of composite cement samples to generate a static physical property dataset; and correlating the static physical property dataset as a function of the dynamic physical property dataset and volume fraction of water to generate a composite cement property model.

Statement 2. The method of statement 1 further comprising: preparing a test cement slurry; measuring a dynamic physical property of the test cement slurry; calculating a static physical property of the test cement slurry using the composite cement property model wherein the dynamic physical property of the test cement slurry is an input to the composite cement property model; and calculating a well life integrity with a numerical simulator using the static physical property of the test cement slurry as and input to the numerical simulator.

Statement 3. The method of any of statements 1-2 wherein the dynamic physical property comprises at least one property selected from the group consisting of dynamic Young's modulus, dynamic Poisson's ratio, dynamic unconfined compressive strength, dynamic tensile strength, dynamic flexural strength, dynamic modulus of elasticity, dynamic shear strength, and combinations thereof.

Statement 4. The method of any of statements 1-3 wherein the static physical property comprises at least one property selected from the group consisting of static Young's modulus, static Poisson's ratio, static unconfined compressive strength, static tensile strength, static flexural strength, static modulus of elasticity, static shear strength, and combinations thereof.

Statement 5. The method of any of statements 1-4 wherein the cement property model has at least one form selected from the group consisting of linear, multilinear, parabolic, exponential, derivative, integral, hyperbolic, trigonometric, and combinations thereof.

Statement 6. The method of any of statements 1-5 wherein the cement property model has at least one form selected from the group consisting of artificial neural network, convolutional neural network, recurrent neural network, decision tree, random forest, machine learning boosting, extreme gradient boosting, Gaussian process regression, spline regression, multi-variate adaptive regression spline, and combinations thereof.

Statement 7. The method of any of statements 1-6 wherein the chemical species comprises at least one species selected from the group consisting of carbon dioxide, ammonia, hydrogen sulfide (H₂S), acid, and combinations thereof.

Statement 8. The method of any of statements 1-7 wherein measuring the dynamic physical property of each of the plurality of composite cement samples comprises measuring the dynamic physical property using an ultrasonic pulse velocity method, acoustic method, a flat-jack test method, or a combination thereof.

Statement 9. The method of any of statements 1-8 wherein measuring the static physical property of each of the plurality of composite cement samples comprises measuring the static physical property using a crushing tests in the presence or absence of confined pressure, a pull out test, a hardness test, a scratch resistance tests, or any combination thereof.

Statement 10. The method of any of statements 1-9 wherein exposing the plurality of set cement samples to the chemical species comprises exposing at least a portion of the set cement samples to differing concentrations of the chemical species.

Statement 11. The method of any of statements 1-10 wherein exposing the plurality of set cement samples to the chemical species comprises exposing at least a portion of the set cement samples to the chemical species for differing amounts of time.

Statement 12. The method of any of statements 1-11 wherein the set cement samples are cured at a pressure in a range of about 1 bar to about 1500 bar and wherein the set cement samples are cured at a temperature in a range of from about 20° C. to about 200° C.

Statement 13. A method comprising: introducing an ultrasonic tool into a wellbore comprising a composite cement sheath wherein the composite cement sheath comprises an unmodified portion and a chemically modified portion; transmitting an ultrasonic wave into the composite cement sheath using the ultrasonic tool; measuring an ultrasonic response using the ultrasonic tool; determining, based at least in part on the ultrasonic response, at least one dynamic cement physical property; and inputting the dynamic cement physical property and a volume fraction of water utilized to prepare the composite cement sheath into a composite cement property model and calculating a dynamic cement physical property.

Statement 14. The method of statement 13 wherein the dynamic physical property comprises at least one property selected from the group consisting of dynamic Young's modulus, dynamic Poisson's ratio, dynamic unconfined compressive strength, dynamic tensile strength, dynamic flexural strength, dynamic modulus of elasticity, dynamic shear strength, and combinations thereof.

Statement 15. The method of any of statements 13-14 wherein the static physical property comprises at least one property selected from the group consisting of static Young's modulus, static Poisson's ratio, static unconfined compressive strength, static tensile strength, static flexural strength, static modulus of elasticity, static shear strength, and combinations thereof.

Statement 16. The method of any of statements 13-15 wherein the cement property model has at least one form selected from the group consisting of linear, multilinear, parabolic, exponential, derivative, integral, hyperbolic, trigonometric, and combinations thereof.

Statement 17. The method of any of statements 13-16 wherein the cement property model has at least one form selected from the group consisting of artificial neural network, convolutional neural network, recurrent neural network, decision tree, random forest, machine learning boosting, extreme gradient boosting, Gaussian process regression, spline regression, multi-variate adaptive regression spline, and combinations thereof.

Statement 18. The method of any of statements 13-17 wherein the chemically modified portion of the composite cement sheath is modified by at least one chemical species selected from the group consisting of carbon dioxide, ammonia, hydrogen sulfide (H₂S), acid, and combinations thereof.

Statement 19. A method comprising: preparing a plurality of cement slurries, wherein the plurality of cement slurries each comprise a cement and volume fraction of water; curing the plurality of cement slurries to form a plurality of set cement samples; exposing the plurality of set cement samples to a chemical species; allowing the chemical species to at least partially modify the plurality of set cement samples to form a plurality of composite cement samples; measuring a dynamic physical property of each of the plurality of composite cement samples to generate a dynamic physical property dataset; measuring a static physical property of each of the plurality of composite cement samples to generate a static physical property dataset; correlating the static physical property dataset as a function of the dynamic physical property dataset and volume fraction of water to generate a composite cement property model; introducing an ultrasonic tool into a wellbore comprising a composite cement sheath wherein the composite cement sheath comprises an unmodified portion and a chemically modified portion; transmitting an ultrasonic wave into the composite cement sheath using the ultrasonic tool; measuring an ultrasonic response using the ultrasonic tool; determining, based at least in part on the ultrasonic response, at least one dynamic cement physical property of the composite cement sheath; and inputting the at least one dynamic cement physical property of the composite cement sheath and a volume fraction of water utilized to prepare the composite cement sheath into the composite cement property model and calculating a dynamic cement physical property of the composite cement sheath.

Statement 20. The method of statement 19 wherein the dynamic physical property comprises at least one property selected from the group consisting of dynamic Young's modulus, dynamic Poisson's ratio, dynamic unconfined compressive strength, dynamic tensile strength, dynamic flexural strength, dynamic modulus of elasticity, dynamic shear strength, and combinations thereof and wherein the static physical property comprises at least one property selected from the group consisting of static Young's modulus, static Poisson's ratio, static unconfined compressive strength, static tensile strength, static flexural strength, static modulus of elasticity, static shear strength, and combinations thereof.

### EXAMPLE

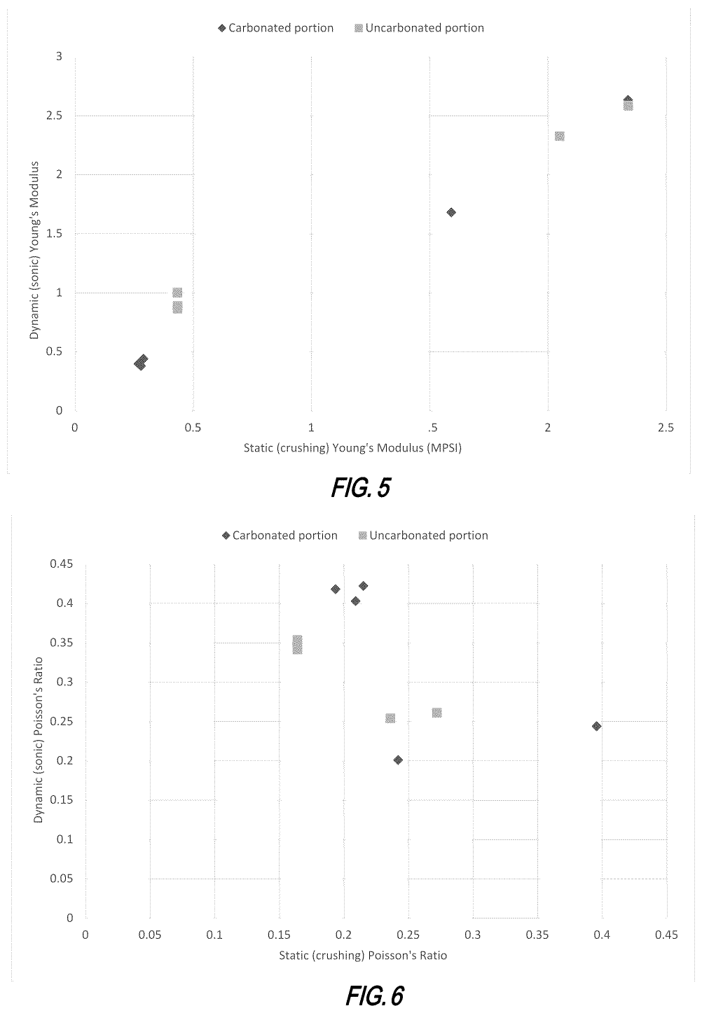

In this example, a carbonated cement property model was constructed as discussed above. Three different cement slurries with different volume fraction of water and composition were prepared and cured at atmospheric pressure and room temperature to form cured cements. Each of the cured cements were subjected to a carbon dioxide environment to carbonate the cured cements. The cured cements were exposed to carbon dioxide for 1 week and 1 month under three different environments, dry, carbonic, and wet where dry CO2 is pure CO2 without water, wet is CO2 in contact with moisture and the cement sample is exposed to this mixture, and carbonic is when CO2 dissolves in water and the cement sample is immersed in water. After carbonation, each of the cured cements was subjected to non-destructive sonic testing using an ultrasonic cement analyzer to measure the dynamic Young's modulus (YM) and dynamic Poisson's ratio (PR). Thereafter, each of the cured cements was subjected to crushing in accordance with API Recommended Practice 10B-2 (Second Edition, April 2013) and/or ASTM D7012-10, to determine the static Young's modulus and static Poisson's ratio. The dynamic and static Young's modulus and Poisson's ratio were plotted. FIG. 5 is a plot of the static Young's modulus to dynamic Young's modulus. FIG. 6 is a plot of the static Poisson's ratio to the dynamic Poisson's ratio. It can be seen in FIG. 5 that the cement slurries with a high volume fraction of water have a distinctly high dynamic Poisson's ratio. The high volume fraction of water cement slurries are clumped together between a dynamic Poisson's ratio of 0.35 to 0.45.

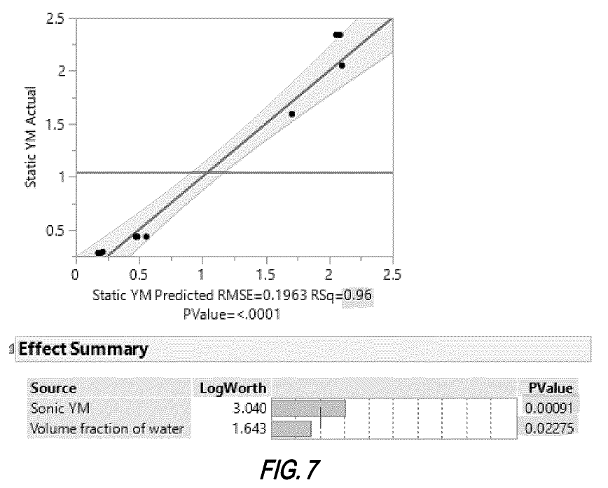

Using the data from FIG. 5, a composite carbonated cement Young's modulus property model was built. The static Young's modulus from crushing was modeled as a function of dynamic Young's modulus from sonic testing and volume fraction of water to form a composite carbonated cement Young's modulus property model. FIG. 7 is a parity plot of the composite carbonated cement Young's modulus property model. The composite carbonated cement Young's modulus property model has a coefficient of determination of 0.96 indicating the model has a good fit for the observed data.

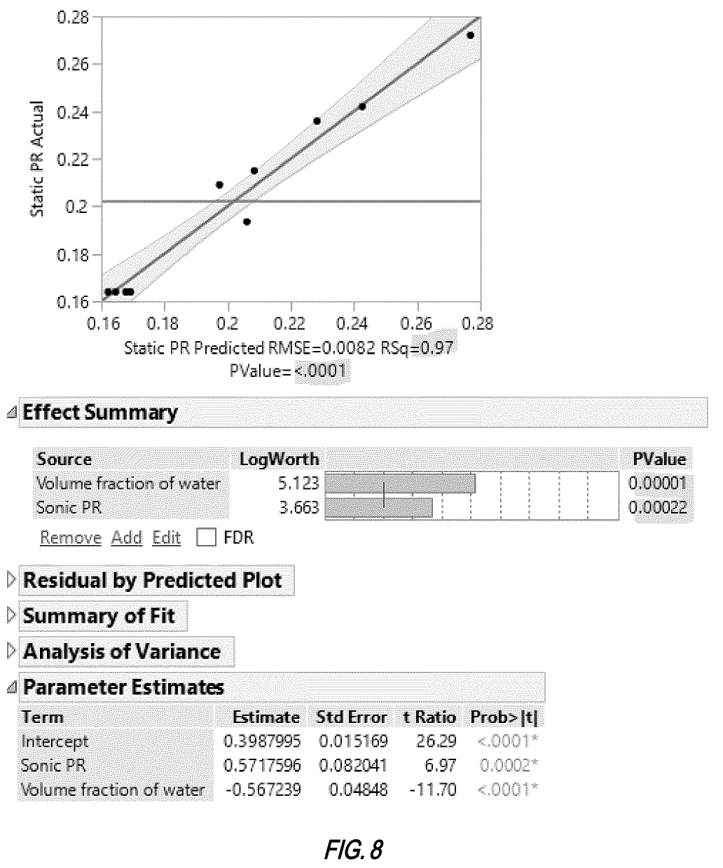

Using the data from FIG. 6, a composite carbonated cement Poisson's ratio property model was built. The static Poisson's ratio from crushing was modeled as a function of dynamic Poisson's ratio from sonic testing and volume fraction of water. FIG. 8 is a parity plot of the composite carbonated cement Poisson's ratio property model. The composite carbonated cement Poisson's ratio property model has a coefficient of determination of 0.97 indicating the model has a good fit for the observed data even with the cement slurries with a high volume fraction of water.

The disclosed cement compositions and associated methods may directly or indirectly affect any pumping systems, which representatively includes any conduits, pipelines, trucks, tubulars, and/or pipes which may be coupled to the pump and/or any pumping systems and may be used to fluidically convey the cement compositions downhole, any pumps, compressors, or motors (e.g., topside or downhole) used to drive the cement compositions into motion, any valves or related joints used to regulate the pressure or flow rate of the cement compositions, and any sensors (i.e., pressure, temperature, flow rate, etc.), gauges, and/or combinations thereof, and the like. The cement compositions may also directly or indirectly affect any mixing hoppers and retention pits and their assorted variations.

It should be understood that the compositions and methods are described in terms of “comprising,” “containing,” or “including” various components or steps, the compositions and methods can also “consist essentially of” or “consist of” the various components and steps. Moreover, the indefinite articles “a” or “an,” as used in the claims, are defined herein to mean one or more than one of the elements that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular examples disclosed above are illustrative only, as the present disclosure may be modified and practiced in different but equivalent manners apparent to those skilled in the art having the benefit of the teachings herein. Although individual examples are discussed, the disclosure covers all combinations of all those examples. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative examples disclosed above may be altered or modified and all such variations are considered within the scope and spirit of the present disclosure. If there is any conflict in the usages of a word or term in this specification and one or more patent(s) or other documents that may be incorporated herein by reference, the definitions that are consistent with this specification should be adopted.

What is claimed is:

1. A method comprising:
preparing a plurality of cement slurries, wherein the plurality of cement slurries each comprise a cement and volume fraction of water;
curing the plurality of cement slurries to form a plurality of set cement samples;
exposing the plurality of set cement samples to a chemical species;
allowing the chemical species to at least partially modify the plurality of set cement samples to form a plurality of composite cement samples;
measuring a dynamic physical property of each of the plurality of composite cement samples to generate a dynamic physical property dataset;
measuring a static physical property of each of the plurality of composite cement samples to generate a static physical property dataset; and
correlating the static physical property dataset as a function of the dynamic physical property dataset and volume fraction of water to generate a composite cement property model.

2. The method of claim 1 further comprising:
preparing a test cement slurry; measuring a dynamic physical property of the test cement slurry; calculating a static physical property of the test cement slurry using the composite cement property model wherein the dynamic physical property of the test cement slurry is an input to the composite cement property model; and calculating a well life integrity with a numerical simulator using the static physical property of the test cement slurry as and input to the numerical simulator.

3. The method of claim 1 wherein the dynamic physical property comprises at least one property selected from the group consisting of dynamic Young's modulus, dynamic Poisson's ratio, dynamic unconfined compressive strength, dynamic tensile strength, dynamic flexural strength, dynamic modulus of elasticity, dynamic shear strength, and combinations thereof.

4. The method of claim 1 wherein the static physical property comprises at least one property selected from the group consisting of static Young's modulus, static Poisson's ratio, static unconfined compressive strength, static tensile strength, static flexural strength, static modulus of elasticity, static shear strength, and combinations thereof.

5. The method of claim 1 wherein the cement property model has at least one form selected from the group consisting of linear, multilinear, parabolic, exponential, derivative, integral, hyperbolic, trigonometric, and combinations thereof.

6. The method of claim 1 wherein the cement property model has at least one form selected from the group consisting of artificial neural network, convolutional neural network, recurrent neural network, decision tree, random forest, machine learning boosting, extreme gradient boosting, Gaussian process regression, spline regression, multi-variate adaptive regression spline, and combinations thereof.

7. The method of claim 1 wherein the chemical species comprises at least one species selected from the group consisting of carbon dioxide, ammonia, hydrogen sulfide (H₂S), acid, and combinations thereof.

8. The method of claim 1 wherein measuring the dynamic physical property of each of the plurality of composite cement samples comprises measuring the dynamic physical property using an ultrasonic pulse velocity method, acoustic method, a flat-jack test method, or a combination thereof.

9. The method of claim 1 wherein measuring the static physical property of each of the plurality of composite cement samples comprises measuring the static physical property using a crushing tests in the presence or absence of confined pressure, a pull out test, a hardness test, a scratch resistance tests, or any combination thereof.

10. The method of claim 1 wherein exposing the plurality of set cement samples to the chemical species comprises exposing at least a portion of the set cement samples to differing concentrations of the chemical species.

11. The method of claim 1 wherein exposing the plurality of set cement samples to the chemical species comprises exposing at least a portion of the set cement samples to the chemical species for differing amounts of time.

12. The method of claim 1 wherein the set cement samples are cured at a pressure in a range of about 1 bar to about 1500 bar and wherein the set cement samples are cured at a temperature in a range of from about 20° C. to about 200° C.

13. A method comprising:
introducing an ultrasonic tool into a wellbore comprising a composite cement sheath wherein the composite cement sheath comprises an unmodified portion and a chemically modified portion;
transmitting an ultrasonic wave into the composite cement sheath using the ultrasonic tool;
measuring an ultrasonic response using the ultrasonic tool;
determining, based at least in part on the ultrasonic response, at least one dynamic cement physical property; and
inputting the dynamic cement physical property and a volume fraction of water utilized to prepare the composite cement sheath into a composite cement property model and calculating a dynamic cement physical property.

14. The method of claim 13 wherein the dynamic physical property comprises at least one property selected from the group consisting of dynamic Young's modulus, dynamic Poisson's ratio, dynamic unconfined compressive strength, dynamic tensile strength, dynamic flexural strength, dynamic modulus of elasticity, dynamic shear strength, and combinations thereof.

15. The method of claim 13 wherein the static physical property comprises at least one property selected from the group consisting of static Young's modulus, static Poisson's ratio, static unconfined compressive strength, static tensile strength, static flexural strength, static modulus of elasticity, static shear strength, and combinations thereof.

16. The method of claim 13 wherein the cement property model has at least one form selected from the group consisting of linear, multilinear, parabolic, exponential, derivative, integral, hyperbolic, trigonometric, and combinations thereof.

17. The method of claim 13 wherein the cement property model has at least one form selected from the group consisting of artificial neural network, convolutional neural network, recurrent neural network, decision tree, random forest, machine learning boosting, extreme gradient boosting, Gaussian process regression, spline regression, multi-variate adaptive regression spline, and combinations thereof.

18. The method of claim 13 wherein the chemically modified portion of the composite cement sheath is modified by at least one chemical species selected from the group consisting of carbon dioxide, ammonia, hydrogen sulfide (H₂S), acid, and combinations thereof.

19. A method comprising:
preparing a plurality of cement slurries, wherein the plurality of cement slurries each comprise a cement and volume fraction of water;
curing the plurality of cement slurries to form a plurality of set cement samples;
exposing the plurality of set cement samples to a chemical species;
allowing the chemical species to at least partially modify the plurality of set cement samples to form a plurality of composite cement samples;
measuring a dynamic physical property of each of the plurality of composite cement samples to generate a dynamic physical property dataset;
measuring a static physical property of each of the plurality of composite cement samples to generate a static physical property dataset;
correlating the static physical property dataset as a function of the dynamic physical property dataset and volume fraction of water to generate a composite cement property model;
introducing an ultrasonic tool into a wellbore comprising a composite cement sheath wherein the composite cement sheath comprises an unmodified portion and a chemically modified portion;
transmitting an ultrasonic wave into the composite cement sheath using the ultrasonic tool;
measuring an ultrasonic response using the ultrasonic tool;
determining, based at least in part on the ultrasonic response, at least one dynamic cement physical property of the composite cement sheath; and
inputting the at least one dynamic cement physical property of the composite cement sheath and a volume fraction of water utilized to prepare the composite cement sheath into the composite cement property model and calculating a dynamic cement physical property of the composite cement sheath.

20. The method of claim 19 wherein the dynamic physical property comprises at least one property selected from the group consisting of dynamic Young's modulus, dynamic Poisson's ratio, dynamic unconfined compressive strength, dynamic tensile strength, dynamic flexural strength, dynamic modulus of elasticity, dynamic shear strength, and combinations thereof and wherein the static physical property comprises at least one property selected from the group consisting of static Young's modulus, static Poisson's ratio, static unconfined compressive strength, static tensile strength, static flexural strength, static modulus of elasticity, static shear strength, and combinations thereof.
